(19) United States
(12) Patent Application Publication
(10) Pub. No.: US 2015/0049177 A1
(43) Pub. Date: Feb. 19, 2015

(54) Camera Arrangement and Image Processing Method for Quantifying Tissue Structure and Degeneration

(71) Applicant: BioOptico AB, Linköping (SE)

(72) Inventors: Anders Johansson, Linkoping (SE); Åke P. Öberg, Ljungsbro (SE); Tommy Sundqvist, Linkoping (SE); Fredrik Persson, Linkoping (SE)

(21) Appl. No.: 14/376,643

(22) PCT Filed: Feb. 6, 2013
(86) PCT No.: PCT/IB2013/050988

§ 371 Date: Aug. 5, 2014

Related U.S. Application Data
(60) Provisional application No. 61/595,300, filed on Feb. 6, 2012.

(51) Int. Cl. A61B 1/317; A61B 1/04; G06T 5/00

(52) U.S. Cl. CPC A61B 1/317; G06T 5/009; A61B 1/04; G06T 2207/10016; G06T 2207/10024; G06T 2207/20172; G06T 2207/30024
USPC 348/71; 382/128

(57) ABSTRACT

Methods and arrangements for detecting osteoarthritis (OA) relate to image processing for enhancing, visualizing and quantifying the fibrillation structure of cartilage using endoscopes. A structure enhancement method comprises obtaining input data, conversion to intensity data, preprocess filtering, intensity fluctuation filtering and contrast enhancement. The degeneration is quantified by a degeneration index (DI) algorithm, applied to the structure enhanced image. Results are then compiled in an output frame presentation.

### TECHNICAL FIELD

The present invention relates to methods and arrangements for detecting osteoarthritis (OA). In particular the present invention relates to image processing for enhancing, visualizing and quantifying the fibrillation structure of cartilage using endoscopes.

### BACKGROUND OF THE INVENTION

Osteoarthritis is the most common type of joint disease, affecting over 20 million individuals in the United States. The condition involves degeneration of articular cartilage and subchondral bone in joints. Typical symptoms include joint pain, stiffness and locking. The processes leading to loss of cartilage are still not fully known, but include a variety of hereditary, metabolic and mechanical factors.

Cartilage is a type of connective tissue, made up of cells (chondrocytes) embedded in a matrix, strengthened with collagen fibers. One of the earliest signs of OA is fibrillation of the collagen structure, seen both as roughening of the cartilage surface and as deeper structure changes. This fibrillation is believed to originate from the breakdown of the collagen fibril network

The fibrillation of cartilage consequently leads to cartilage softening and, with time, deeper cartilage defects. At this stage the condition becomes visible arthroscopically, but the earlier stages are not visible in arthroscopy or in any other clinical imaging technique, maybe with the exception of MRI where some recent progress has been made.

Endoscopic techniques have been used for the diagnosis and therapy of disorders since the beginning of the twentieth century. One typical example is arthroscopy, where the interior of a joint is visualized. Arthroscopy is primarily a diagnostic procedure but is also performed to evaluate or to treat many orthopaedic conditions such as torn cartilage, damaged menisci or ruptured ligaments.

The arthroscope provides visual information from the interior of a joint. Demands have been raised, though, that a more quantitative approach would improve the quality of diagnosis and therapeutic decisions, as well as serve as a tool in education and in patient communication. To assess whether the cartilage is normal, abnormal or absent is of particular interest in these situations.

To assess whether the cartilage is absent or present can be made by a cartilage thickness measurement approach. Clinical standards for this include magnetic resonance or radiographic methods, often in combination with image analysis. Of relevance in endoscopy are methods that can be utilized during the surgical intervention. In situ cartilage has for instance been studied with ultrasonic methods (SAARAKALA et al., 2006, VIRÉN et al., 2009), but of particular interest are methods based on optical measurement, as the system components of an endoscopic set-up can be modified and used for the purpose Important examples include spectroscopic (JOHANSSON et al., 2011, JOHANSSON et al., 2012, KINNUNEN et al., 2010, ÖBERG et al., 2004) and optical coherence based approaches (CHU et al., 2007, DREXLER et al., 2001, HERRMAN et al., 1999).

When cartilage is present, it is important to assess whether it is normal or abnormal with respect to fibrillation structure. Today this is primarily performed using histology on cartilage biopsies (PASTOUREAU et al, 2003). In clinical routine in situ, assessment is made visually and by probing the cartilage. Early structural changes are not, however, visually detectable and the assessment also depends on the experience of the operating surgeon.

### SUMMARY OF THE INVENTION

The current invention describes an optical method for enhancing and visualizing the fibrillation structure of cartilage. The imaging results can also be reduced to objective measures that quantify degeneration. Main application is in arthroscopical assessment of OA. In this set-up, the image processing method for enhancing tissue structure and the algorithm for quantifying tissue degeneration can be implemented in an endoscopic camera. Existing cameras can be used, with care taken on where to locate the algorithms in the video processing path. In addition to cartilage, the invention may also be useful in assessing other intra-articular structures during arthroscopy, such as menisci or ligaments.

The current invention describes image processing means for enhancing and visualizing the fibrillation structure of cartilage, as well as an algorithm for quantifying tissue degeneration. The calculations are made by an endoscopic camera, with care taken on where to locate the image processing algorithms in the video processing path. According to described procedures, the structure enhancement algorithm consists of obtaining input data, conversion to intensity data, preprocess filtering, intensity fluctuation filtering and contrast enhancement. The degeneration is quantified by a degeneration index (DI) algorithm, applied to the structure enhanced image. Results are then compiled in an output frame presentation.

Thanks to the invention it is possible to provide means for automatic image enhancement of the cartilage structure and for objective quantification of the degeneration.

### DESCRIPTION OF THE DRAWINGS

FIG. 1 shows the different parts of the structure enhancement and degeneration index algorithms.

FIG. 2 shows an endoscopic video camera set-up, with the algorithms for structure enhancement and degeneration quantification located in the video processing path.

FIG. 3 shows examples from an ex vivo study on knee condyles, removed from patients (n=11) undergoing total knee replacement because of osteoarthritis. Left image shows a normal cartilage surface after application of the structure enhancement algorithm. Right image shows corresponding image from an osteoarthritic cartilage region.

FIG. 4 shows results from a clinical study on routine knee arthroscopy patients (n=33). In the study structure was enhanced and degeneration indices were calculated from 33 sites of normal cartilage and from 58 degenerated cartilage sites. The figure shows mean±standard deviation. The difference was statistically significant (p<0.05).

FIG. 5 shows examples of applying structure enhancement and degeneration index calculation to images of reference sandpaper surfaces of varying degrees of surface roughness. Higher degeneration index values were seen for higher degrees of roughness, corresponding to higher degrees of tissue degeneration.

FIG. 6 shows a normal arthroscopical view of the cartilage and the cartilage surface (left). Right part of figure shows the same view, after the structure enhancement algorithm has been applied. The osteoarthritic cartilage fibrillation structure is enhanced and visualized. Note that in this example the enhancement algorithm has only been applied to bright pixels, leaving darker pixels untreated.

### DETAILED DESCRIPTION

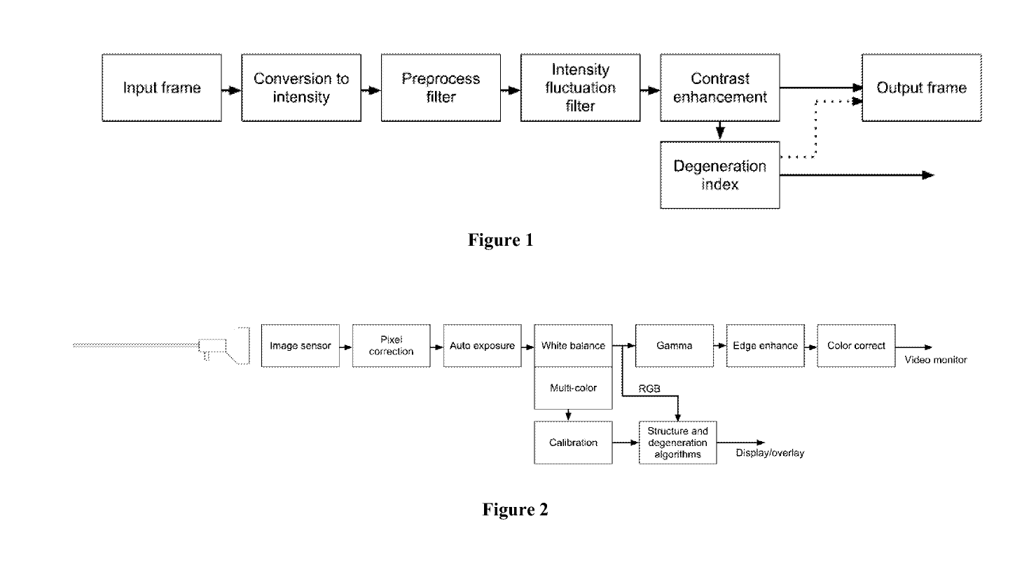

The main steps of the tissue structure enhancement method according to the invention can be summarized as: Obtaining input data, conversion to intensity data, preprocess filtering, intensity fluctuation filtering, contrast enhancement and output frame presentation (FIG. 1). These different steps are described below, together with a description of the DI calculation.

### Obtaining Input Data

Circuitry and processing within an endoscopic video camera are well suited to perform algorithm calculations and graphically present the result as an enhancement to the live arthroscopic image. For purposes of displaying the best endoscopic image on the surgical monitor, the raw red, green and blue (RGB) signals collected by the camera and endoscope are, in endoscopic cameras used today, modified by both linear and non-linear transformations. For example, edge enhancement, color correction and gamma correction. Such transformations may affect the quality of algorithm calculations. On the other hand, automatic exposure, white balance, and defective pixel correction are camera processes applied to the RGB signals that improve the repeatability and quality of the calculations. Given these constraints, FIG. 2 shows where in the video processing path the RGB signals are taken for input to the algorithm formulas shown below. The calculations are performed in a field programmable gate array (FPGA) for each pixel in every video frame.

### Conversion to Intensity Data

There are indications that some tissues undergo spectral changes during degeneration, for instance cartilage during OA progression, but in the most specific solution, the enhancement algorithm uses only intensity data. The RGB data from the input frame is therefore reduced to a single intensity frame, preferably by calculating the mean value of the red, green and blue channel values of the input frame. An alternative solution is to select one of the three channels. This selection influences the tissue level at which the structure is enhanced.

### Preprocess Filtering

The structure enhancement algorithm is based on local fluctuations in intensity, caused by the light interacting with the fibrillated tissue, leading to tissue structure dependent fluctuations in the back-scattered light. Partly to bring out the faint details in these fluctuations, covered by noise, and partly to adjust to the desired level of fibrillation to enhance, a preprocessing filter is applied. This is typically an averaging or Gaussian low-pass filter. Filter size and other characteristics are chosen depending on image resolution, tissue type and what level of the fibrillations to enhance. A typical choice for arthroscopic 960×540 pixel video/image assessment of degenerated cartilage is a 10×10 averaging filter.

### Intensity Fluctuation Filtering

The central part of the structure enhancement algorithm is the application of a local intensity fluctuation enhancement operator. This is typically performed by using a standard image filtering approach with a specific X×Y pixel kernel. The kernel can be applied to the preprocessed image pixel by pixel or in a stepwise manner, for instance to reduce computing demands. The calculation can be done in separable horizontal and vertical steps. The kernel calculation is based on deriving a single measure related to intensity variation, for instance variance (Equation 1), standard deviation, entropy (Equation 2) or some other statistical measure of variation.

$${I}_{se} = \frac{1}{N} {\sum }_{i = 1}^{N} {( {P}_{i} - μ )}^{2} \qquad ( 1 )$$

Here Iₛₑ is the kernel output value describing structure enhanced values, N the number of kernel values, Pᵢ the kernel pixel values and μ the kernel pixel average value.

Iₛₑ=−Σᵢ₌₁^{N} Hᵢ log Hᵢ (2)

Here H is the histogram of the kernel pixel values.

Kernel size depends on the same geometrical and tissue dependent factors as in the preprocessing step, but a typical example for arthroscopic 960×540 pixel video/image assessment of degenerated cartilage is to use a 5×5 variance or standard deviation based kernel.

The output image from this processing step will be referred to as the structure enhanced image.

### Contrast Enhancement

If visualizing the structure enhanced image, a contrast enhancement may be appropriate. This could include mapping the result onto the dynamic range [0 255] according to Equation 3.

I_{ce}=255(Iₛₑ−t₁)(t₂−t₁) (3)

Here I_{ce} is the contrast enhanced image and the t values are contrast level thresholds.

The output image from this processing step will be referred to as the contrast enhanced image.

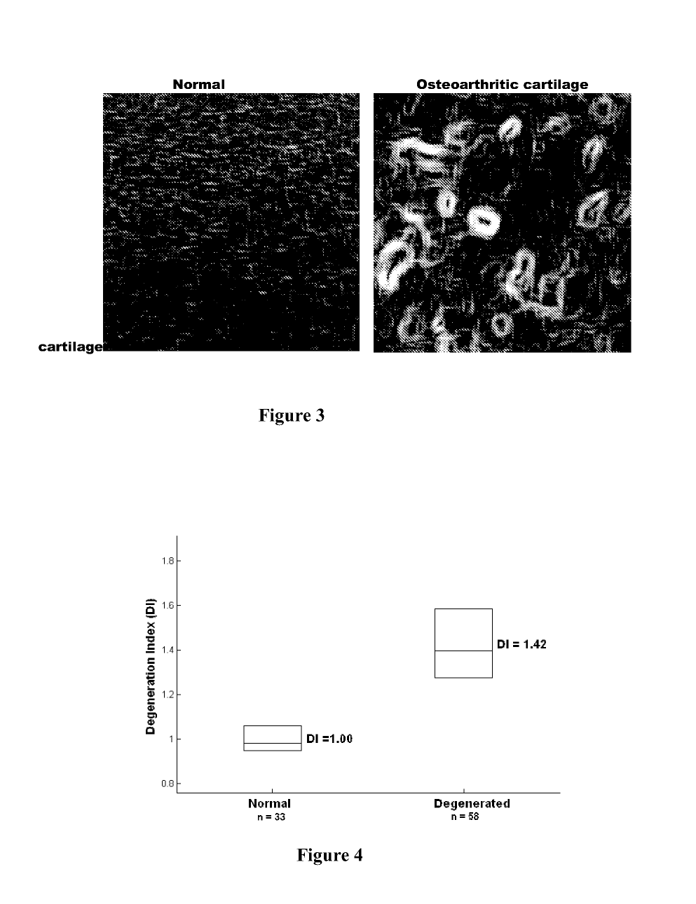

In FIG. 3 examples of contrast enhanced images are shown. The images are from an ex vivo study on knee condyles, removed from patients (n=11) undergoing total knee replacement because of OA. Left image shows a normal cartilage surface after application of the structure enhancement algorithm. Right image shows corresponding image from an OA cartilage region.

### Degeneration Index Calculation

The local or global pixel values in the structure enhanced image, before or after contrast enhancement, can be reduced to a single DI value based on variance analysis. More advanced approaches include pattern recognition or Fourier domain analysis to quantify pixel fibrillation.

The DI makes it possible to quantitatively compare different degeneration stages of tissue. In FIG. 4 results from a clinical study on routine knee arthroscopy patients (n=33) are shown. Here, DI values were calculated using standard deviation of structure enhanced images, derived from 33 sites of normal cartilage and from 58 degenerated cartilage sites. The figure shows mean±standard deviation. The difference was statistically significant (p<0.05).

### Output Frame Presentation

Generating an output frame based on the structure or contrast enhanced output images can be made in many different ways. One example is using a picture in picture approach, where the processed image is presented together with the input frame; another is showing the processed result as an overlay to the input frame. In the latter example the output image values are applied to selected regions of the input image. Regions can for instance be those that are not too bright because of over exposure, too dark because of insufficient illumination, or where the derived output image values give rise to a local DI value that is higher than a specific threshold. Worth noting is that the overlay may consist of the enhanced output image values themselves or be presented in a simplified fashion using a specific colour or a colour according to a look-up-table.

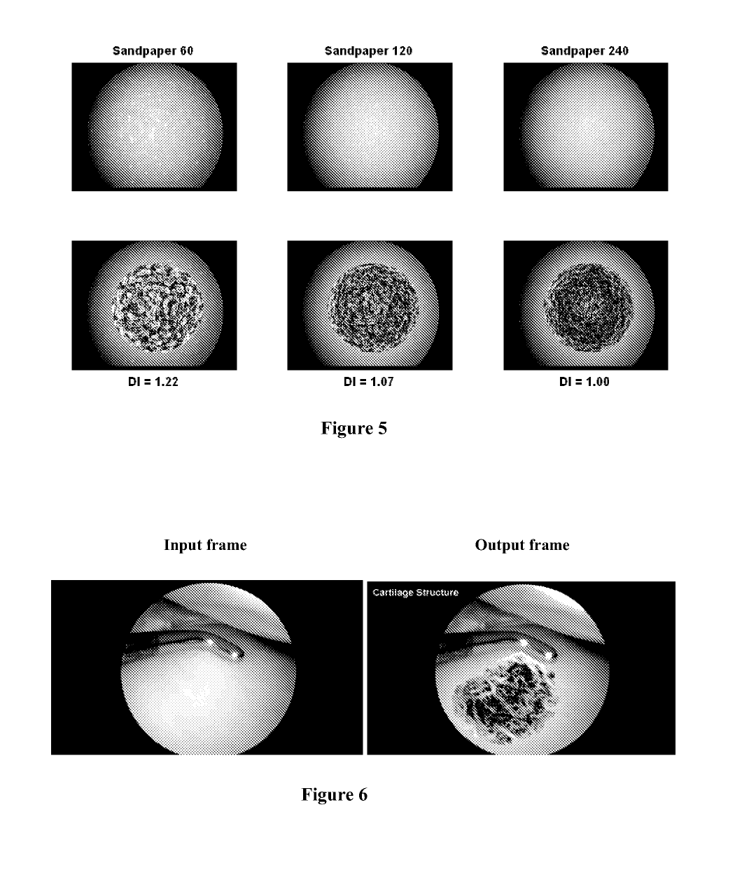

Image examples are shown in FIG. 5-6. FIG. 5 shows examples of applying structure and contrast enhancement, followed by DI calculation, to images of sandpaper surfaces of varying degrees of surface roughness. Higher DI values are seen for higher degrees of roughness, corresponding to higher degrees of tissue degeneration.

In left part of FIG. 6, a normal arthroscopical view of cartilage is presented. In the right part of the figure, the same view is seen after the structure and contrast enhancement algorithms have been applied. The OA cartilage fibrillation structure is enhanced and visualized. In this example the enhancement algorithm has only been applied to bright pixels, leaving darker pixels untreated.

### Additional Comments

The method and arrangement according to the present invention has been described as performed within a camera. This is a convenient solution. However, as understood by the skilled person, the necessary calculations can be performed in any suitable equipment, such as external computers or dedicated external devices. Such solutions can for instance be attractive if to use older types of cameras, or for off-line image processing applications.

The current description of the invention is also focused on the processing of still images captured by a camera or other device. There is an obvious possibility to present processed images in video sequences, off-line or on-line during surgery. Furthermore, there may be value in basing the image processing or output presentation not only on single frames, but also on cross-frame statistics or variables.

### REFERENCES

Chu C R, Ferretti M, Studer R K: Clinical diagnosis of potentially treatable early articular cartilage degeneration using optical coherence tomography. J Biomed Opt. 2007; 12(5).
Drexler W, Stamper D, Jesser C, et al.: Correlation of collagen organization with polarization sensitive imaging of in vitro cartilage: Implications for osteoarthritis. J Rheumatol. 2001; 28:1311-8.
Herrman J M, Pitris C, Bouma B E et al.: High resolution imaging of normal and osteoarthritic cartilage with optical coherence tomography. J Rheumatol. 1999; 26:627-35.
Johansson A, Sundqvist T, Kuiper J-H, Öberg P Å: A spectroscopic approach to imaging and quantification of cartilage lesions in human knee joints. Phys Med Biol. 2011; 56:1865-78.
Johansson A, Kuiper J-H, Sundqvist T, et al.: Spectroscopic measurement of cartilage thickness in arthroscopy: Ex vivo validation in human knee condyles. J Arthroscopic Rel Surg. 2012; 28:1513-23.
Kinnunen J, Vahimaa, P, Jurvelin J S, et al.: Optical spectral imaging of degeneration of articular cartilage. J Biomed Opt. 2010; 15.
Pastoureau P, Leduc S, Chomel A, De Ceuninck F: Quantitative assessment of articular cartilage and subchondral bone histology in the meniscectomized guinea pig model of osteoarthritis. Osteoarthritis Cartilage. 2003; 11(6):412-23.
Saarakkala S, Laasanen M S, Jurvelin J S, Töyräs J: Quantitative ultrasound imaging detects degenerative changes in articular cartilage surface and subchondral bone. Phys Med Biol. 2006; 51(20):5333-46.
Virén T, Saarakkala S, Kaleva E, et al.: Minimally invasive ultrasound method for intra-articular diagnostics of cartilage degeneration. Ultrasound Med Biol. 2009; 35(9):1546-54.
Öberg P Å, Sundqvist T, Johansson A: Assessment of cartilage thickness utilising reflectance spectroscopy. Med Biol Eng Comput. 2004; 42:3-8.

1. An endoscopic video camera device, for deriving structure enhanced images of internal body structures and tissues, the camera device comprising image processing means adapted to:
obtain input frame RGB data,
convert the input frame RGB data to intensity frame data,
pre-process the intensity frame data,
filter the pre-processed frame data with an intensity fluctuation filter, resulting in a structure enhanced image,
enhance the contrast of the structure enhanced image, and
generate an output frame.

2. The device according to claim 1, wherein said conversion of input frame RGB data to intensity frame data includes selecting one colour channel or averaging the three colour channels of the camera.

3. A device according to claim 2 where said pre-processing of intensity frame data includes a Gaussian or an averaging image low-pass filter.

4. A device according to claim 3 where said intensity fluctuation filtering includes an image filter kernel based on variance, standard deviation or entropy.

5. A device according to claim 4 where said contrast enhancement includes mapping the structure enhanced image to a specific dynamic range.

6. A device according to claim 5 where said output frame is generated as a picture in picture, including input frame data and structure or contrast enhanced frame data.

7. A device according to claim 6 where algorithm invocation is based on a particular user input such as pressing a camera head button.

8. A device according to claim 5 where said output frame is generated using an overlay approach, where input frame data is replaced by processed data.

9. A device according to claim 8 where said replacement is based on thresholds for too dark input data pixels, too bright input data pixels or degeneration index values.

10. A device according to claim 9 where the pixels of the input frame data are replaced by corresponding values from the structure enhanced frame data, the contrast enhanced frame data, a specific colour or colors according to a look-up table.

11. A device according to claim 10 where algorithm invocation is based on a particular user input such as pressing a camera head button.

12. A method for deriving structure enhanced images of internal body structures and tissues, comprising the steps of:
obtaining input frame RGB data,
converting the input frame RGB data to intensity frame data,
pre-processing the intensity frame data,
filtering the pre-processed frame data with an intensity fluctuation filter, resulting in a structure enhanced image,
enhancing the contrast of the structure enhanced image, and
generating an output frame.

13. The method for deriving structure or contrast enhanced images according to claim 12, further comprising the step of calculating a tissue degeneration index, based on the structure or contrast enhanced image.
